(19) United States
(12) Patent Application Publication
(10) Pub. No.: US 2010/0268224 A1
(43) Pub. Date: Oct. 21, 2010

(54) BIPOLAR ELECTROSURGICAL TOOL WITH ACTIVE AND RETURN ELECTRODES SHAPED TO FOSTER DIFFUSE CURRENT FLOW IN THE TISSUE ADJACENT THE RETURN ELECTRODE

(76) Inventors: Trevor Landon, Kalamazoo, MI (US); Jonathan O. Thorne, Boulder, CO (US)

Correspondence Address: INTEL. PROP./ RND;STRYKER CORPORATION, 4100 EAST MILHAM AVE., KALMAZOO, MI, 49001-6197, US

(21) Appl. No.: 12/425,959

(22) Filed: Apr. 17, 2009

(51) Int. Cl. A61B 18/18

(52) U.S. Cl. 606/42; 606/48; 606/50

(57) ABSTRACT

A bipolar electrosurgical tool including a shaft to which active and return electrodes are mounted. The return electrode is mounted to the shaft and shaped to have an exposed surface that, in at least one direction has a substantially linear profile. A bore extends through the return electrode and opens into the portion of the exposed surface with the substantially linear profile. The active electrode is seated in the bore and extends forward from the exposed surface. A collar formed from electrically insulating material extends over the portion of the active electrode adjacent the return electrode. The collar extends both outwardly of the active electrode and forward of the exposed surface of the return electrode.

### FIELD OF THE INVENTION

This invention relates generally to a bipolar electrosurgical tool such as the type of tool used to cut tissue and thereafter coagulate the cut tissue.

### BACKGROUND OF THE INVENTION

An electrosurgical tool is a surgical tool with features designed to flow current through tissue adjacent the tool. Some electrosurgical tools are designed to cut tissue. In this process, the tool, more particularly, at least one electrode integral with the tool, is positioned adjacent the tissue to be cut. Current is flowed from/to the electrode. This current flows through the tissue adjacent the electrode. Owing to the resistance of the tissue, the current, electrical energy, is converted into thermal energy. This thermal energy heats the tissue to a level at which the liquid within the cells forming the tissue vaporizes. The rapid expansion of this liquid within the tissue-forming cells causes the cells to burst. The bursting of the cells is what causes the separation, the cutting, of the tissue.

An electrosurgical tool can also be used to coagulate tissue. When an electrosurgical tool is used to coagulate tissue, a smaller amount of current is usually flowed through the tissue than when the tool is used to cut the tissue. This smaller current flow causes the tissue to heat less than when the tissue is cut. As a consequence of this heating, the proteins in the tissue forming the cell undergo a state change. Also, the fluid internal to the cells more slowly boils off in comparison to the rapid boiling during the cutting process. Collectively, the transformation of the cell proteins and the slow boiling of the cell fluids solidifies the tissue-forming cells. This mass of solidified material forms a barrier that prevents leakage of fluids, such as blood, from the underlying tissue.

An electrosurgical tool can be provided with an electrode that is relatively small in size. One small sized electrosurgical tool is a micro-dissection needle. This type of electrosurgical tool can have an electrode with a length of 2.5 mm or less and a cross sectional area of 0.15 mm² or smaller. When an electrosurgical tool with this size electrode is operated in the cutting model, only a very small section of tissue, the tissue disposed against the face of the electrode, is cut. An electrosurgical tool with an electrode having the above characteristics can be used to make very fine cuts in or resections of tissue. These cuts are much more difficult to make, or in some cases impractical to make, using mechanical, steel blade surgical cutting instruments.

For many years, medical practitioners relied on monopolar electrosurgical tools to perform electrosurgical cutting and coagulation procedures. A monopolar surgical tool system includes a tool with a single electrode, a conductive ground pad and a control console. Both the tool and the ground pad are connected to the control console. At the start of the procedure, the ground pad is placed in contact with the skin of the patient. During the procedure, the control console drives current between the tool electrode and the ground pad. The tool electrode has a much smaller surface area than the ground pad. Accordingly, the current flow is most dense in the tissue adjacent the tool electrode. Depending on the characteristics of the current flow, the current through the tissue adjacent the tool electrode causes thermal energy to be generated resulting in cutting and/or coagulation of the tissue.

In recent years, bipolar electrosurgical tools have become popular. A bipolar electrosurgical tool is a tool that includes complementary active and return electrodes. One bipolar electrosurgical tool is disclosed in the Applicants' U.S. patent application Ser. No. 11/146,867, published as U.S. Pat. Pub. No. US 2005/0283149 A1, the contents of which are explicitly incorporated herein by reference. This tool of this document is a cutting tool that has an active electrode with a much smaller surface area than the return electrode. The above incorporated by reference bipolar surgical tool, like many bipolar surgical tools, is designed so that the exposed surface of the active electrode emerges directly from the exposed face of the return electrode.

By appropriately driving the current to/from the active electrode of an electrosurgical tool, the adjacent tissue can be heated in such a manner that, as the tissue is cut, the tissue is coagulated.

Bipolar electrosurgical tools are useful for cutting tissue and essentially simultaneously minimizing the bleeding of the cut tissue. However, there are some disadvantages associated with this type of surgical instrument. Small bits of tissue have been known to catch or stick around the base of the active electrode, the location where the active electrode emerges from the return electrode. These bits of tissue can form conductive bridges between the electrodes that extend over the insulator between the electrodes. Initially, when such a bridge is formed, the bridge functions as short circuit through which substantially all the current between the electrodes flows. Since most of the current flow is through this short circuit, the tissue cutting and tissue coagulating current flow, at least momentarily, essentially ceases.

As consequence of the short circuit flow through the trapped bit of tissue, the trapped tissue rapidly coagulates into char. This char adheres to the base of the active electrode and the adjacent surface of the return electrode. The char around the active electrode impedes the current flow to/from the underlying surfaces of the active electrode. The impedance of the current flow to/from the char covered portion of the active electrode results in a like reduction in the cutting and coagulation of the tissue around that char.

Also, as mentioned above, a bipolar electrosurgical tool is typically constructed so that the return electrode has a relatively large exposed surface. However, often the tool is shaped so that, when the return electrode is pressed against the tissue against which a procedure is to be performed, only a small section of the return electrode contacts the tissue. Often, geometrically, this section of the electrode has a circular cross-sectional shape. Given the relatively small area of the tissue-electrode contact, relatively dense currents have been known to flow through this tissue. These dense current flows have been known to cause tissue heating that results in undesirable transformation of the tissue. In some situations, the current density through this tissue can be so high that tissue is heated to the level at which the cells forming the tissue burst. In other words, the current flow around the return electrode can be so high that it causes the removal or damage of the tissue that practitioner wanted left at the site at which the procedure is being performed.

Current density through the tissue surrounding the return electrode can be especially high when the electrode is initially pressed again the tissue. This is because, when a return electrode is initially pressed against the tissue, the electrode presents a very small circular contact area, essentially a point contact, to the tissue. Immediately after this contact, as the return electrode is continued to be pushed against the tissue, the surface area of this interface, the diameter or the circle, increases. Nevertheless, initially the surface area of this interface is quite small. In some circumstances, this surface area can even be less than that at which the active electrode has in contact with the tissue. At this time, if the tool is active, the density of the current flowing through the tissue adjacent this interface can be very high. The current flowing through the tissue is therefore especially prone to heat the tissue to levels that cause the cells forming the tissue to undergo undesirable transformations.

Furthermore, as discussed above, tissue trapped between the active and return electrodes of a bipolar surgical tool can form char around the portion of the return electrode against which the tissue is trapped. This char reduces the low impedance surface area of the tissue-return electrode interface through which current readily flows. The reduction of the surface area of this interface results in a corresponding increase in the density of the current flow through tissue forming the interface. Again, this current density can reach a level sufficient to cause tissue and electrode heating that, in turn, causes undesirable changes in the tissue.

### SUMMARY OF THE INVENTION

This invention is directed to a new and useful bipolar electrosurgical tool. The tool of this invention includes features designed to increase the density of the current flow in the tissue surrounding the active electrode and minimize the density of the current flow in the tissue adjacent the return electrode.

The electrosurgical tool of this invention includes a return electrode with an exposed front surface that is large in width. An active electrode emerges from the exposed front surface of the return electrode. An insulating collar extends over the base of the active electrode, the section of the active electrode that emerges from the return electrode. This collar reduces the likelihood that caught tissue forms a conductive bridge between the electrodes.

Owing to the return electrode having a relatively large width, the return electrode presents a relatively large surface against the tissue to which tool is applied. Consequently, when the electrosurgical tool is pressed against tissue, the current density through the tissue forming the tissue-return electrode interface is relatively low. The low density of this current flow minimizes the extent to which this current causes undesirable changes in the cells forming this tissue.

In some versions of the invention, the active electrode is formed to have a cross sectional profile wherein one face has a relatively short width and a second face adjacent the first face has a longer width. When the tool is used to cut tissue, the short width face is pressed against the tissue to function as the cutting face of the electrode. This construction and use of the invention results in a very high current flow through the tissue forming the electrode cutting face-tissue interface.

### BRIEF DESCRIPTION OF THE DRAWINGS

The invention is pointed out with particularity in the claims. The above and further features and benefits of the invention are explained in the following Detailed Description taken in conjunction with the accompanying drawings in which:

FIG. 1 is a perspective view of a pencil-type bipolar electrosurgical tool of this invention;

FIG. 2 is a plan view of the top of the electrosurgical tool of FIG. 1;

FIG. 3 is a plan view of the side of the electrosurgical tool of FIG. 1;

FIG. 4 is a cross sectional view of the electrosurgical tool taken along line 4-4 of FIG. 3;

FIG. 5 is a perspective view of the return electrode of the tool of FIG. 1;

FIG. 6 is a side plan view of the return electrode of FIG. 5;

FIG. 7 is a cross sectional view of the return electrode of FIG. 5;

FIG. 8 is an enlarged cross sectional view of the distal end of the tool of FIG. 1 when viewed along the longitudinal axis;

FIG. 9 is an enlarged perspective view of distal end of the tool of FIG. 1;

FIG. 10 is a perspective view of an alternative electrosurgical tool constructed in accordance with this invention;

FIG. 11 is a side plan view of the tool of FIG. 10;

FIG. 12 is a cross sectional view of the tool of FIG. 10;

FIG. 13 is a perspective view of the sub-assembly that forms the active electrode of the tool of FIG. 10;

FIG. 13A is a partial cross sectional view of the active electrode-forming sub-assembly of FIG. 13;

FIG. 14 is a perspective view of the return electrode of tool of FIG. 10;

FIG. 15 is a side view of the return electrode of FIG. 10;

FIG. 16 is a cross sectional view of the return electrode of FIG. 10;

FIG. 17 is a perspective view of the distal end of the tool of FIG. 10;

FIG. 18 is a cross sectional view of the distal end of the tool of FIG. 10 viewed along the longitudinal axis of the tool;

FIG. 19 is a perspective view of the distal end of an alternative pencil-type electrosurgical tool of this invention;

FIG. 20 is a side view illustrating the use of the electrosurgical tool of FIG. 19;

FIG. 21 is a perspective view of the distal end of an alternative loop-type electrosurgical tool of this invention; and

FIG. 22 is a side view illustrating the use of the electrosurgical tool of FIG. 21.

### DETAILED DESCRIPTION

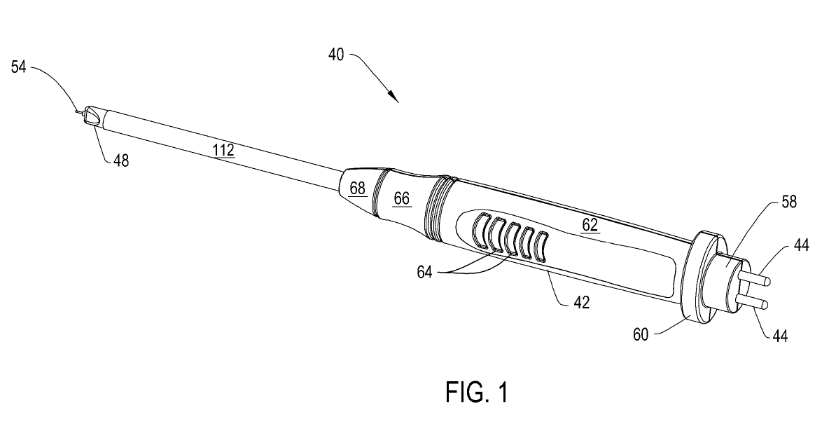

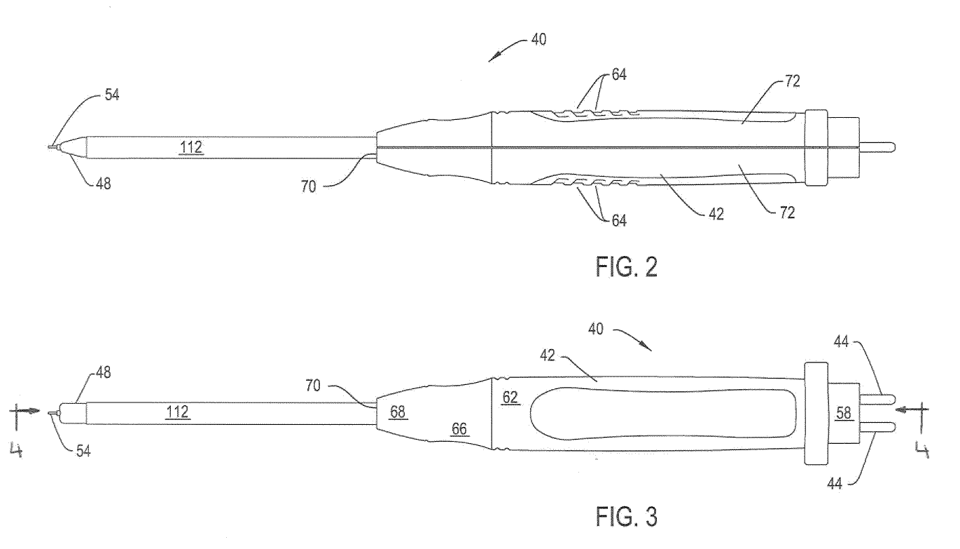

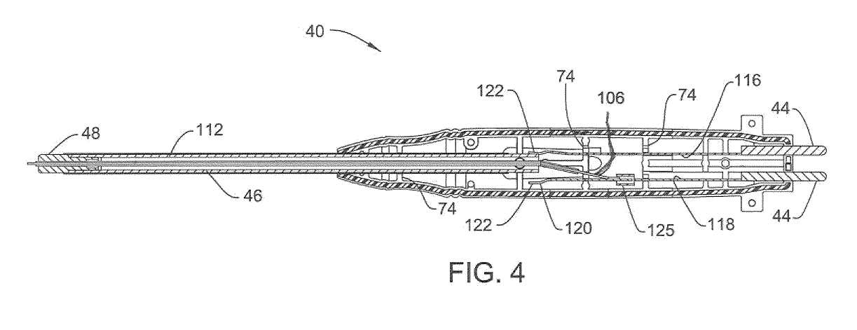

FIGS. 1-4 depict a pencil type bipolar electrosurgical tool 40 constructed in accordance with this invention. Tool 40 includes a handle 42. Two conductive terminals 44 extend in parallel rearwardly out of the proximal end of the handle 42. (“Proximal” is understood to be towards the practitioner holding the tool 40, away from the surgical site to which the tool is applied. “Distal” is understood to be away from the practitioner, towards the site to which the tool 40 is applied.) An elongated shaft 46 extends forward from the distal end of the handle 42. A return electrode 48 is seated in the open distal end of shaft 46. Return electrode 48 has an exposed front surface 86 (FIG. 9) located forward of shaft 46. An insulating shell 112 extends over shaft 46 and the adjacent end of the return electrode 48. A thin, cylindrical active electrode 54 extends forward from return electrode front surface 86.

Handle 42 is generally in the form of a multi-section tube. In the illustrated version of the invention, the handle 42 is shaped to have a base 58 that forms the proximal end of the handle. Handle base 58 has a constant outer diameter. A waist 60 is located immediately forward of base 58. Waist 60 has an outer diameter along its length that is constant and greater than the outer diameter of base 58. While not readily apparent in the drawings, in many versions of the invention, waist 60 has a non-circular cross sectional profile, (in the plane perpendicular to the longitudinal axis of handle 42). This non-circular profile reduces the extent that, when the tool 40 is placed on a flat surface, the tool will roll. Forward of waist 60, handle 42 has a main section 62. In terms of length, main section 62 is the longest section of the handle 42. Main section 62 has a outer diameter that is slightly greater than that of the base 58 and less than that of the waist 60. While the outer diameter of handle main section 62 is generally constant, the main section may be provided with one or more sets of longitudinally spaced apart ribs 64 (not seen in FIG. 3). The ribs are defined by indentations in the main section 62 (indentations not identified). Ribs 64 serve as finger holds for the practitioner. Not identified are two grooves that extend annularly around the forward end of the handle main section 62.

Forward of main section 62, handle 42 has a neck 66. Neck 66 has an outer diameter that varies along the length of the neck. Extending distally forward of the main section 62, the outer diameter of the neck tapers inwardly. At a position approximately two/thirds along the length of the neck forward from the proximal end, the outer diameter of the neck starts to increase to the distal front end of the neck. The diameter of the neck 66 at the proximal end is greater than that at the distal end. The neck 66 is further shaped so that the varying diameter of the neck gives the neck along a longitudinal slice section thereof a concave profile.

Handle 42 has a head 68 that is the most forward section of the handle. The head 68 is located forward of neck 66. Head 68 has a frusto-conical shape such that the diameter of the head decreases distally from the neck 66. An annular groove, seen in FIG. 1 and not identified, is located between the handle neck 66 and head 68. The distal front end of handle head 68 is formed with a circular opening 70.

In some versions of the invention, handle 42 is formed from polystyrene or other non-conductive plastic. Handle 42 may also be formed from two shells 72. Internal to the shells 72 are support ribs 74. The support ribs 74 are formed with slots and grooves, not identified, in which the components of tool 40 internal to the handle 42 are seated.

Shaft 46 is a tube of aluminum or other electrically conductive material. In some versions of the invention, shaft 46 has an outer diameter of between 2.0 to 6.0 mm in most versions of the invention and in many versions of the invention between 3.5 to 4.5 mm. Shaft 46 has a wall thickness of between 0.5 to 2.0 mm and more often between 1.0 and 1.5 mm. Approximately 15 to 50% of the proximal most section of the shaft 46 by length is disposed in handle 42. This portion of the shaft 46 is compression fitted in the grooves formed in the support ribs 74 internal to handle 42. The shaft 46 extends forward out of the handle 42 through head opening 70.

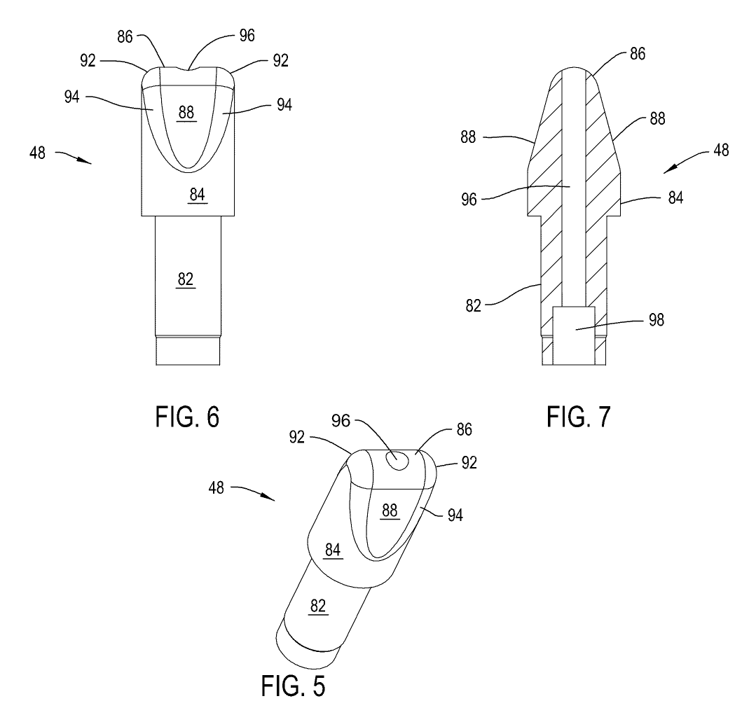

Return electrode 48 is formed from a solid piece of electrically conductive material to which tissue does not readily stick or adhere. A metal having a relatively high thermal conductivity/diffusivity often has this characteristic. In some versions of the invention, the return electrode 48 is formed from silver or an alloy of silver. As seen in FIGS. 5-7, return electrode 48 is shaped to have a cylindrical tail 82 that forms the proximal end of the electrode. Tail 82 has an outer diameter that allows the tail to be interference fitted/secured in the open end of shaft 46. The proximalmost one-fifth end of tail 82 has a diameter slightly less than the forward four-fifths of the tail. This is to facilitate assembly of tool 40 and is otherwise not relevant to this invention.

Forward of tail 82 return electrode 48 has a head 84. Immediately forward of tail 82, head 84 has a cylindrical shape. The outer diameter of this portion of return electrode head 84 is approximately equal to the outer diameter of shaft 46. Electrode head 84 is further shaped to so that the most forward surface, front surface 86, has, in lateral cross section, has an arcuate shape. In cross section, surface 86 subtends an arc of between 90 and 180°. In some version of the invention, tool 40 is shaped so that, in cross section surface 86 has a radius of curvature of at least 0.5 mm and, in many versions of the invention, at least 0.7 mm. Front surface has a length, the distance along the longitudinal axis of surface 86, of at least 0.5 mm and more particularly at least 1.0 mm. This distance, it should be understood, is the distance along a line.

Two opposed side surfaces 88 taper outwardly away from the opposed sides longitudinally extending sides of electrode front surface 86. Each side surface 88 angles away from the adjoining proximal end of the front surface 86 by an angle of angle of between 10 and 20° (measured from a datum parallel to the longitudinal axis of the tool 40) and more often between 12 to 18°. Below front surface 86 and between the side surfaces 88, the electrode head 84 has curved outer surfaces 94 that are extensions of the cylindrical outer surface at the proximal end of the head. Collectively, the portion of the electrode head 84 that defines the front surface 86, side surfaces 88 and outer surface 94 typically extend over at least 50% of the total length of the electrode head 84. In some versions of the invention, these surfaces of the electrode 48 may extend over the whole of the length of head 84.

Electrode head 84 is further shaped so that a curved corner surfaces 92 functions as the transition surfaces between each end of the front surface 86, the adjacent distal ends of outer surfaces 94 and circular outer wall of the head 84. The circumferential length and radius of curvature of each corner surface 92 is greatest where the surface 94 is closes to the end of the adjacent front surface. Corner surfaces 94 meet at the proximal most ends of each side surface 88. At the locations where the corner surfaces 94 meet, surfaces 94 have their shorts arcuate length and radius of curvature.

Return electrode 48 is further formed to have a bore 96 that extends longitudinally therethrough. Bore 96 has a distal end opening (not identified) in the electrode head front surface 86. The return electrode 48 is further formed so a counterbore 98 that is concentric with bore 96 extends forward from the distal end of tail 82. In the illustrated version of the invention, counterbore extends approximately one-third through the tail 82 from the proximal end of the tail. Bores 96 and 98 are concentric with the longitudinal axis through the return electrode 48.

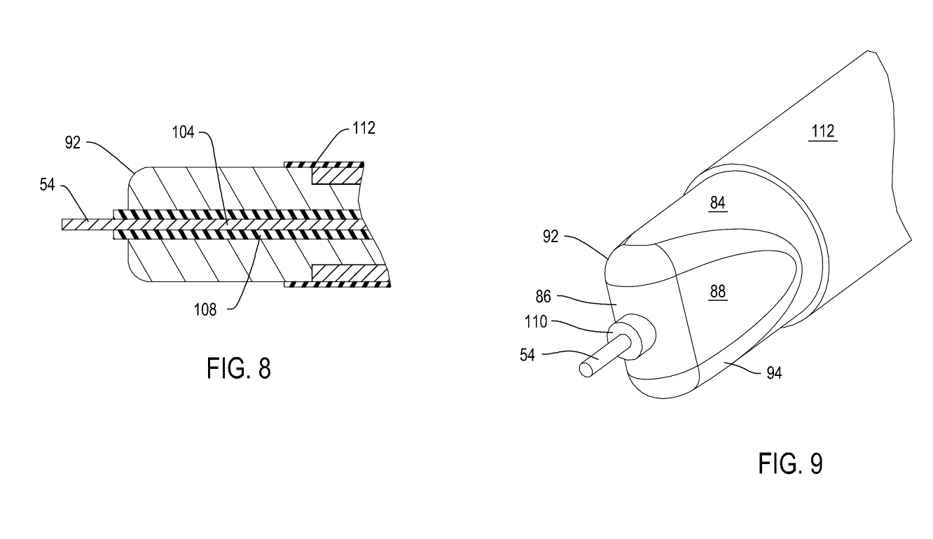

Active electrode 54 is part of a thin, cylindrical, electrically conductive rod 104 (FIG. 8). Often this material forming at least electrode 54 if not the whole of rod 104 has a lower thermal conductivity/thermal diffusivity than the material forming return electrode 48. In one version of the invention, rod 104 is formed from tungsten. Rod 104 has a length greater than the combined length of the shaft-return electrode sub-assembly. The diameter of rod 104 is less than that of return electrode bore 96. Rod 104 extends both through the lumen of shaft 46 and bore 96 of the return electrode 48. More particularly, when tool 40 is assembled, rod 104 has a distal end section that extends forward at least 1.0 mm from the return electrode 48. Typically, rod 104 extends forward from the return electrode 48 a maximum distance of 7.0 mm and often, 3.0 mm or less. This distal end section of the rod 104 is active electrode 54. Rod 104 also has a proximal end section 106 disposed in handle 42 that extends approximately 2 cm rearward from the proximal end of shaft 46.

A tube 108 formed from electrically insulating material is disposed over most of, but not all of rod 104. In some versions of the invention, tube 108 is a PTFE tube that is heat shrunk over the rod 104. Insulating tube 108 extends over the sections of rod 104 disposed in shaft 46 and in the return electrode. Insulating tube 108 also extends a short distance forward electrode front surface 86 so as to be disposed around the active electrode 54. This exposed portion of the insulating tube is identified in the Figures as insulating collar 110. Insulating collar 110 extends forward over the base of the active electrode 54 from the exposed return electrode front surface 86 a distance between 0.1 and 1.5 mm and, more often between 0.1 and 1.0 mm. Insulating collar 110 has a wall thickness of between 0.1 and 0.5 mm. Insulating tube 108 also extends over most of the section of rod 104 that extends rearward from shaft 46. The insulating tube 108 does not extend over the most proximal 2.5 to 10 mm of rod 104.

The outer tubular shell 112 is disposed over shaft 46. Shell 112 is formed from an electrically insulating material such as flouropolymer that is heat shrunk over the shaft 46. The shell 112 extends forward from a position approximately 0.5 cm forward of the proximal end of the shaft. Shell 112 extends outside over the whole of the portion of shaft 46 located outside of the handle 42. The shell 112 also extends forward over a short distance over the adjacent return electrode head 84. In some versions of the invention, shell 112 extends 1 to 4 mm over the proximalmost portion of the return electrode head 84.

Each terminal 44 is part of a contact 116 mounted to the handle 42. Each contact 116 is a single piece of conductive metal such as stainless steel. The proximal portion of each contact 116 has a cylindrical shape. This portion of the contact 116 is the terminal 44. Not identified is the rounded proximal end of the terminal. A thin section of metal, a leg 118, extends distally forward from the distal front end of the terminal 44. Leg 118 is able to flex. Each contact 116 is further shaped to have an ankle 120 that extends rearwardly from the distal end of the leg. The ankle 120 extends both forward and angularly away from the leg 120. A foot 122 extends forwardly away from the distal end of the ankle 120. Foot 122 is bent relative the ankle. More particularly, each contact 116 is formed so that the foot 122, while offset from the associated leg 118, is approximately parallel to the leg 118.

When the tool 40 of this invention is assembled, the shaft subassembly is often disposed in one of the handle-forming shells 72. Here, “shaft subassembly” is understood to mean the shaft 46, the return electrode 48, rod 104, tube 108 and shell 112. As seen in FIGS. 8 and 9, the tube-encased rod 104 tightly fits in return electrode bore 96. Shell 112 is tightly disposed over the shaft 46 and the adjacent end of the return electrode head 84. Contacts 116 are seated in the same shell 72. More particularly, each contact 116 is seated in the shell 72 so that the terminal 44 integral with the contact extends out of a semi-circular opening (not identified) in the proximal end of the shell 72. The leg 118 of one of the contacts is bent towards the leg of the opposed contact. Owing to the bending of the leg, the foot 122 of the bent-leg contact 116 abuts the adjacent outer surface of shaft 46 to establish physical and electrical connections between the contact 116 and the shaft.

Also in the assembly process, the proximal end of the tube-covered rod 104 is bent so that proximal to where the rod emerges from shaft 46, the rod angles toward the other contact 116. A crimp 125 or solder weld establishes a physical mating and electrical connection between the rod 104 and the leg 118 of the second contact 116.

Once the connectors-to-shaft subassembly connections are made, the second shell 72 is mounted and attached to the component-holding shell 72. This completes the assembly of tool 40.

Electrosurgical tool 40 of this invention is readied for use in the same manner in which a conventional bipolar electrosurgical tool is readied for use. A cable is connected between the tool terminals 44 and a power console (not illustrated and not part of this invention). The power console includes a power generator able to generate the signals that result in the sequential flow of currents between the electrodes 48 and 54 that result in the cutting and coagulation of tissue.

Tool 40 is employed to cut tissue by actuating the power console. The tool 44 is placed against the tissue to be cut. In this step, the active electrode 54 is initially positioned at the location at which the tissue is to be cut. Immediately after the active electrode 54 is pressed against the tissue, the return electrode front surface 86 presses against the tissue. As a consequence of both electrodes 48 and 54 pressing against the tissue, there is current flow between the electrodes, through the tissue. Owing to the relatively small exposed surface area of the active electrode 54, the density of current flow is densest in the tissue around this electrode 54. Consequently this is the tissue that is heated to the level at which, first, the cells forming the tissue initially burst to form cuts in the tissue. The tissue-forming cells exposed by the cutting are then heated to a level at which they coagulate.

During this process, at least the front surface 86 of the return electrode 48 and often one of the corner surfaces 92 are pressing against tissue. Consequently, there is current flow through the tissue against these surfaces, that is, the tissue against which the return electrode is pressed. The return electrode thus presents a surface area that, geometrically, is similar to a rectangle against the adjacent tissue. In comparison to a tool with a return electrode that presents a circle to the adjacent tissue, the surface area of this electrode-tissue interface is relatively large. Consequently, there is relatively low density current flow through the tissue forming this interface. This current flow is typically of such low density that the flow does not cause an unwanted effect in the tissue in contact with or adjacent the return electrode 48.

A low density current flow is even present through the tissue adjacent the return electrode 48 during the beginning of the cutting procedure. This is the moment during the procedure in which the return electrode 48 initially presses against the tissue. This is because even at this initial step of the cutting process, owing to the shape of the return electrode 48, the front surface 84 initially presents a rectangular surface area, against the tissue. While initially this surface area may be relatively small, short in length and an even shorter in width, it is greater than the almost point-like surface area a conventional return electrode may initially present to the tissue.

While there may not be as much heating of the tissue abutting the return electrode as there is heating of the tissue adjacent the active electrode, the tissue adjacent the return electrode is still heated. Owing to the return electrode being formed from material having a high thermal diffusivity/thermal conductivity, the thermal energy stored in this tissue is conducted away from the face of the return electrode, towards the distal end of the tool 40. The rapid conduction of this heat away from the surface of the return electrode 48 further minimizes the instances of the tissue adjacent the return electrode heating to the level at which the tissue is damaged.

Another feature of tool 40 of this invention is that insulating collar 110 presents an insulating surface between the return and active electrodes 48 and 54, respectively, that has a relatively wide distance between the electrodes. This distance has both a horizontal aspect, the lateral distance between the two electrodes 48 and 54, and a vertical aspect, the distance along the length of the active electrode 54 forward of the return electrode 48. During a procedure, bits of tissue may catch between the active electrode 54 and the insulating collar 110. Bits of tissue may also catch between the return electrode 48 and the insulating collar 110. However, owing to the separation between the electrodes 48 and 54 established by the insulating collar 110 and that this separation has both a horizontal and vertical aspect, tissue caught in the active electrode-insulating collar interface is held forward of the return electrode 48. Similarly, tissue caught in the return electrode-insulating collar interface is held laterally away from the active electrode 54. Consequently, it is unlikely that a bit of tissue trapped against either electrode 48 or 54 will extend to the other electrode 54 or 48. The significant reduction of the caught tissue, which is conductive, bridging electrodes 48 and 54 reduces the instances of the problems caused by the formation of these bridges.

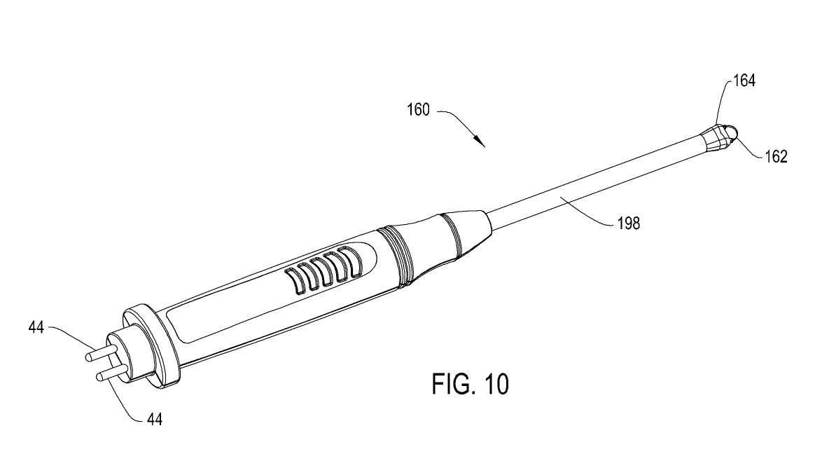

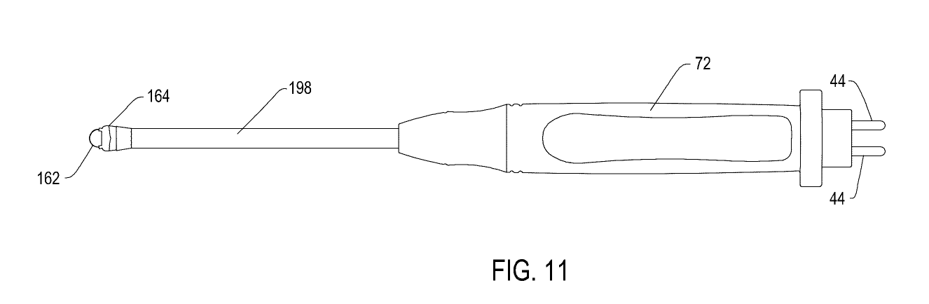

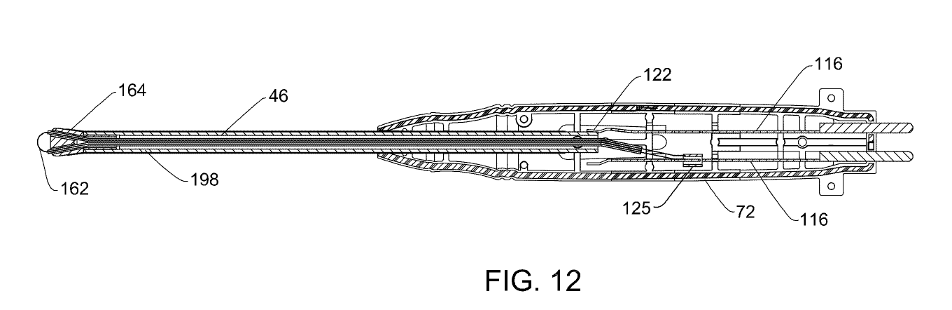

FIGS. 10-12 illustrate an alternative bipolar electrosurgical tool 160, a loop tool, constructed in accordance with this invention. Tool 160 includes the handle 42, shaft 46 and terminal-forming contacts 116 incorporated into tool 40. Tool 160 also includes a loop-type active electrode 162 and a complementary return electrode 164.

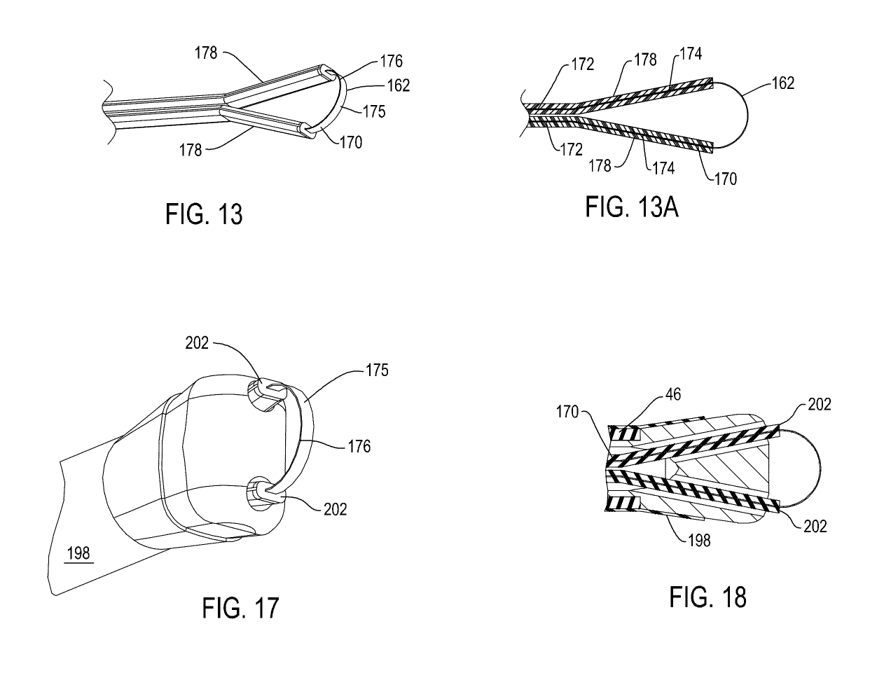

Active electrode 162 is part of an elongated, folded-over rod 170, partially seen in FIGS. 13 and 13A. Rod 170 is formed from the material from which rod 104 is formed. In cross-section, rod 170 has a rectangular shape. In some versions of the invention, the cross-sectional length across rod 170 is between 0.2 and 0.8 mm and often between 0.4 and 0.6 mm. The cross sectional width of the rod 170 is between 0.01 and 0.1 mm. Generally, the ratio, length to width, of a cross sectional slice through rod 170 is between 1:1 and 40:1 and, more often, between 8:1 and 12:1.

During the assembly of tool 160, rod 170 is bent to have two parallel legs 172. Legs 172 have a length greater than that of shaft 46. At the distal end of the tool 160, each leg 172 transitions into a foot 174. Feet 174 angle away from the longitudinal axis of shaft 46 in opposed directions. The feet 174 bend outwardly from the legs 172 along equal and opposite angles. Between the feet 174, rod 170 has an arcuate section that subtends an arc of at least 180°. This arcuate section of rod 170 and the distal ends of the rod feet 174 from which this arcuate section extends can be considered the active electrode 162 of tool 160. The opposed parallel long length sectional surfaces of rod 170 that are part of the active electrode 162 can be considered to be the major faces 175 of the electrode. The opposed parallel short length width faces of the active electrode 162 are the minor faces 176. In FIGS. 13 and 17, only the outwardly directed, the distally directed major face 175 of the active electrode is seen. A single minor face 176 is also seen in these Figures. In these Figures, owing to the relatively short distance across the minor face 176, the minor face while having some depth, essentially appears to be an arcuate slice of a circle.

When tool 160 is assembled, the opposed ends of the folded-over rod 170 extend out of the opposed ends of shaft 46. The proximal ends of the legs 172 are located rearward of the proximal end of shaft 46. Crimp 125 holds the proximal ends of the legs 172 of rod 170 to one of the leg 118 of one of the contacts 116. The distal ends of the legs 172, feet 174 and active electrode 162 are located forward of the distal end of the shaft 46.

Insulating tubes 178 are disposed over substantially all of each of the rod legs 172 and feet 174. Insulating tubes 178 are formed from the same material from which insulating tube 108 is formed. Each insulating tube 178 starts at a location forward of proximal end of the rod leg 172 over which the tube is disposed. Each insulating tube 178 covers the foot 174 associated with the leg 172 covered by the tube.

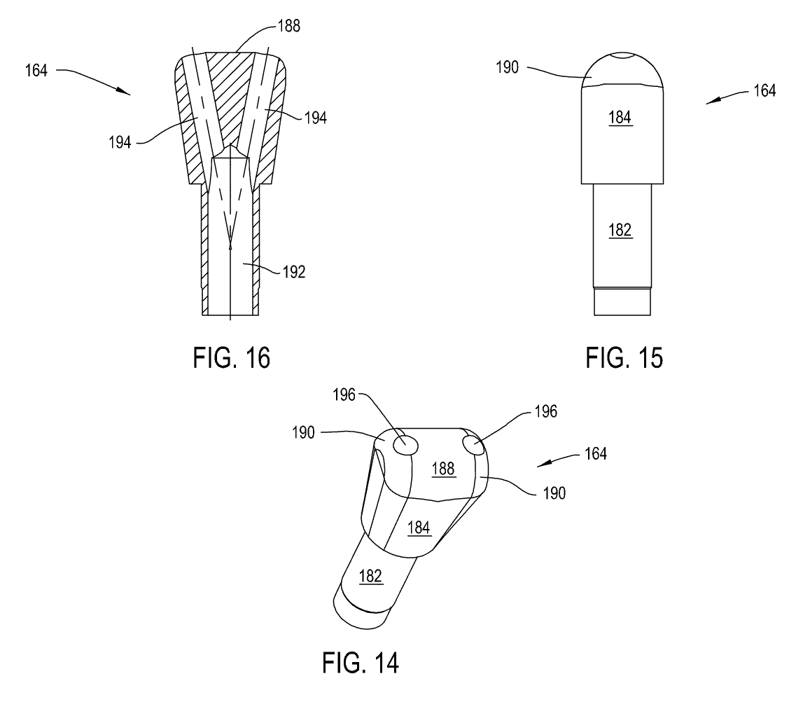

Return electrode 164 of electrosurgical tool 160 can be formed from the same material from which return electrode 48 of tool 40 is formed. As seen by reference to FIGS. 14-16, return electrode 164 is shaped to have a cylindrical tail 182. Tail 182 is dimensioned to fit in the open distal end of the lumen of shaft 46. Forward of tail 182, return electrode 164 has a neck 184. Generally, neck 184 can be described in cross section as having the shape of a rectangle with rounded corners. Return electrode 164 is further shaped so that, as neck 184 extends forward from tail 182, the cross-sectional length of the neck increases. At the most distal end, neck 184 has a length, the distance between the minor surfaces, that is approximately 1.9 times greater than the diameter of the electrode tail 182.

Forward of neck 184, return electrode is shaped to have a head 186. Head 186 has a front surface 188 that has an arcuate shape such that the opposed major side edges of the front surface curve between the opposed major side surfaces of neck 184. Front surface 188 subtends an arc of 180° and has a radius of curvature of at least 0.5 mm and more often at least 1.5 mm. Front surface 188 has a length, as measured along an axis parallel to the axis between the minor surfaces of neck 184, of between 0.5 and 10 mm. Electrode front surface 188 has opposed ends. Each end is spaced inwardly from an adjacent minor surface of the electrode neck 184. Between each end of the front surface 188 and adjacent neck minor surface, there is a curved corner surface 190. Each corner surface 190 curves outwardly and rearwardly to function as the transition surface between each end of the front surface and the adjacent neck minor surface.

The return electrode 164 is formed to have a bore 192 that extends forward from the proximal end of tail 182. Bore 192 has a diameter that allows the bore to receive the tube-encased distal end of legs 172. Bore 192 extends axially through the electrode tail 182 and a short distance into neck 184. The distal end of bore 192 opens into two branch bores 194 that extend through the electrode neck 184. Branch bores 194 diverge from bore 192 along equal and opposite angles relative to the longitudinal axis of bore 192. Each branch bore 194 opens into the electrode front surface 188. More particularly, the front surface opening 196 of each branch bore partially intersects one of the ends of the front surface 188 and the adjacent electrode corner surface 190. Each branch bore 194 has a diameter that allows the bore to tightly receive one of the tube-covered feet 174 of rod 170. In the version of the invention illustrated in FIGS. 14-18, return electrode 164 is formed so that openings 196 are in the most distal portion of front surface 188.

A tubular shell 198 essentially identical to shell 112, is disposed over shaft 46. Shell 198 can be formed from the same material from which shell 112 is formed. Shell 198 extends over the same portion of shaft 46 disposed in handle 42 over which shell 112 extends. Tool 160 is further constructed so that shell 198 extends forward of the distal end of shaft 46 and a short distance over the rear portion of return electrode neck 184.

When tool 160 is assembled, the tube-covered legs 172 of rod 170 are disposed in the lumen of shaft 46. The distal ends of legs 172 are disposed in return electrode bore 192. Each tube-covered foot 174 of rod 170 is disposed in a separate one of the branch bores 194. The distal end of each tube covered foot 174 of rod 170 extends a short distance, out of one of the openings 196 in the return electrode 164. The arcuate portion of the rod 170 that partially forms the active electrode 162 arcs forward of return electrode front surface 188. More particularly, rod 170 is mounted in the return electrode 162 so that the outermost face of active electrode, is one of the major faces 175. The active electrode minor faces 176 are in separate parallel planes that are parallel with and extend on opposed sides of the longitudinal axis of shaft 48. The minimum spacing between the most forward portion of the active electrode rearwardly directed major face 175 and the return electrode front surface is 0.5 mm. The maximum spacing between the most forward portion of the active electrode distally directed major face 175 and the return electrode front surface 188 is usually a maximum 10 mm and more often 6 mm or less. The distal end portions of the tubes 178 that extend forward of the return electrode extend forward from the front surface of the return electrode by the same distance with which collar 110 extends forward from the front surface of the return electrode 48 of the first described version of the invention. These portions of tubes 178 are called out in FIG. 17 as insulating collars 202 that extend over the proximal ends of active electrode 162, the ends of the active electrode closest to the return electrode 164.

When bipolar electrosurgical tool 160 of this invention is used, the practitioner positions the tool so that one of the active electrode minor faces 176 and the return electrode 164 are pressed against the tissue to be cut or resected. Current to/from the active electrode 162 passes across the minor face 176 pressed against the tissue. This minor face 176 presents a relatively small surface area to the tissue. Current density through the tissue on this side of this active electrode-tissue interface is therefore relatively high. This high-density current flow through the tissue of this interface facilitates the rapid cutting and coagulation of the tissue. It should therefore be appreciated that the active electrode minor faces 176 across which this current is flowed functions as the cutting face of the electrode 162.

While the distance across the minor face 176 of the active electrode performing the cutting may be relatively short, the distance across the adjacent major faces 175 of the electrode are relatively large. This relatively large width across the active electrode 162 provides mechanical strength to the electrode. This mechanical strength minimizes the likelihood that, when pressed against the tissue, the active electrode will deflect, or worse yet, fracture.

Return electrode 164 of this version of the invention has a geometric profile similar to that of the return electrode 48 of tool 40. Consequently, when pressed against tissue return electrode 164 presents a relatively wide surface to the tissue, even when the electrode initially contacts the tissue. This keeps the density of the current flow through the tissue forming this interface with the return electrode low so as to reduce the problems associated with high current flow through this tissue.

Electrosurgical tool 160 of this invention further includes insulating collars 202 disposed around the opposed base ends of the active electrode 162; the ends of the active electrode that emerge from the surfaces of the return electrode. The size and vertical and horizontal aspect of these collars reduces the likelihood that tissue caught around either one of the electrodes 162 or 164 will form a bridge to the other electrode 164 or 162. The reduction in the incidence of the formation of these bridges results in a like reduction in the problems short conductive bridges have been known to cause.

The above description is directed to two versions of the electrosurgical tool of this invention. Other versions of the tool of this invention may have features different from what has been described. Thus, the dimensions and materials called out in this disclosure are merely exemplary unless recited in the claims.

Similarly, there is no reason that all versions of the invention have each of the above described features. Thus, in some versions of the invention, it may be desirable to omit either the return electrode having the described geometry or the insulating collars that are disposed around the bases of the active electrodes.

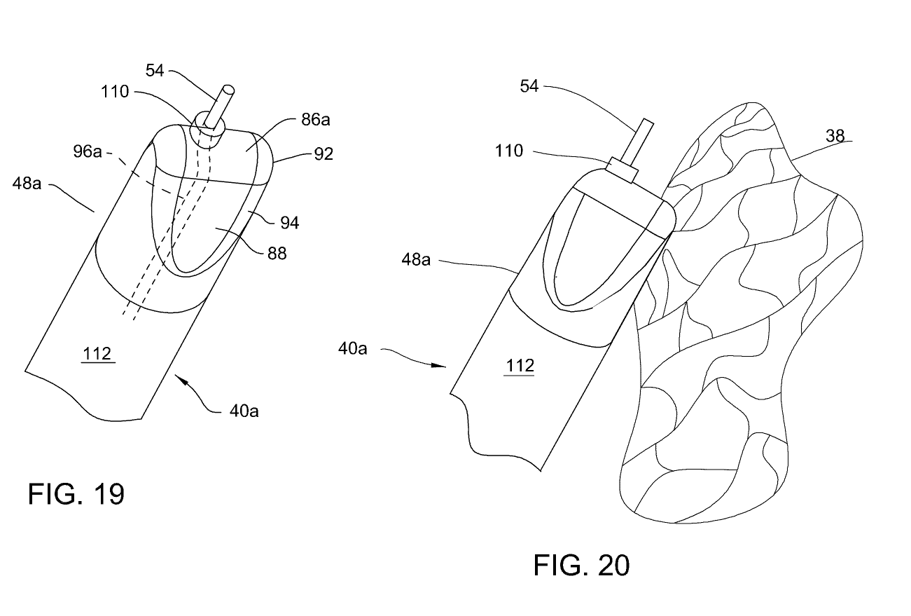

Likewise the electrodes of this invention may not have the symmetric arrangements as described an illustrated above. FIG. 19 for example illustrates the distal end of an alternative tool 40a of this invention. Tool 40a has a return electrode 48a similar to the return electrode 48 off FIGS. 5-7. However, return electrode 48a is formed with a bore (not illustrated that is parallel to though laterally spaced from the longitudinal axis through the electrode. Active electrode 54 and collar 110 thus emerge from the front surface 86a of electrode 48a at a location spaced from the longitudinal axis of return electrode 48a.

The advantage of this asymmetric design is seen in FIG. 20. Specifically this construction of the invention allows tool 40a to be placed against tissue so that, initially, return electrode abuts the tissue 38 to which the tool is applied. This ensures that, when the active electrode 54 abuts the tissue, current will immediately flow through the tissue, between the electrodes. This reduces the likelihood that, during the initial application of the tool to tissue, diffuse current to/from the active electrode could reduce the desired heating of the tissue to the level at which the tissue is cut. Moreover, this design minimizes the extent to which the surface of the return electrode 48a that does not function as the portion of the electrode 48a through which current is flowed obstructs the view around the active electrode 54.

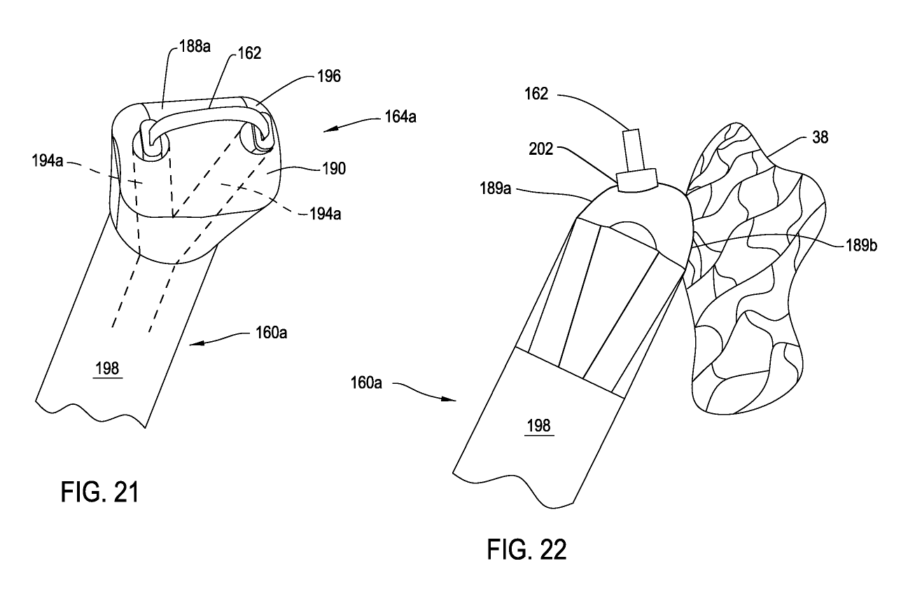

FIG. 21 illustrates the distal end of a loop-type electrosurgical tool 160a designed to likewise minimize the diffusion of current during the initial contact of the tool to tissue 38. Tool 160a has a return electrode 164a similar to electrode 164 of FIGS. 14-16. Return electrode 164a is however constructed so that the bores from which the ends of active electrode 162 do not emerge from the distal most portion electrode front surface 188a. Instead, the ends of the active electrode 162 and surround collars 202 emerge from bores (not illustrated) that are spaced slightly proximally from the distalmost portion of front surface 188a.

As seen in FIG. 22 this allows tool 160a to be placed against tissue 38 so that the first portion of an electrode that strikes the tissue are the surface of the return electrode on the side of the longitudinal axis of the return electrode opposite the side from which active electrode 162 emerges. When the active electrode 162 then strikes the tissue 38, the current will immediately flow through the tissue. This essentially eliminates the disadvantages associated with having a more diffuse current flow to/from the active electrode which can occur if it is the first electrode to strike the tissue.

FIG. 22 also illustrates a further feature that may be incorporated into both the pencil and loop type electrode array assemblies of this invention. Specifically, the return electrode 194 may be designed so that the side 189a of front surface 188a from which the active electrode emerges has a larger radius of curvature, or more pronounced taper than the oppose side 189b. An advantage of this version of the invention is that it increases the visibility to the practitioner around the active electrode, especially where the active electrode emerges from the return electrode.

Also, the features of the two versions of the invention may, if required, be interchanged. Thus, one could have a pencil-type electrosurgical tool with an active electrode having the rectangular profile of tool 160. Similarly, a loop type tool may have an active electrode with a circular cross sectional profile.

Likewise the basic structure shapes of both the active and return electrodes may be different from what has been described. Thus, the active electrode, either the pencil type or the loop type, may not seat in the plane through which the longitudinal axis of the adjacent distal end of the return electrodes extend. In other words, the active electrodes of this invention, either at the location of where they emerge from the return electrodes or distal to this location may be angled relative to the extension of the longitudinal axis through the adjacent distal end of the return electrode. Similarly, there is no requirement that in all versions of the invention, the return electrodes be part of sub-assemblies that simply extend linearly from the handle. In some versions of the invention, the return electrodes may be angled relative to the longitudinal axis of the proximal section of the associated shaft, the section of the shaft that extends from the handle.

Furthermore it should be understood that while in many versions of the invention it is preferred that the slice section of the return electrode from which the active electrode emerges by absolutely linear, it is not required. In many versions of the invention it is merely desired that the slice section of the return electrode from which the active electrode emerges by substantially linear. Here, “substantially” linear should be understood to mean having a radius of curvature of at least 3.8 mm, if not at least 10 mm. This ensures that when the return electrode does contact tissue, the contact will be over a relatively wide area so as to ensure the diffuse current flow through the tissue.

While the handle of this invention as shown as pencil shaped, this is likewise not a requirement for this invention. In some versions of the invention, to accommodate the preferences of some practitioners, the handle may, for example, be in the shape of a pistol so as to have both a grip and a barrel from which the shaft extends. In some versions of the invention, the handle may simply be an insulated proximal end portion of the shaft.

It should likewise be understood that this invention is not limited to the described pencil and loop type electrosurgical tools. This invention may be incorporated into electrosurgical tools with electrodes having shapes other than the described rod (pencil electrode) or loop.

There is no requirement that in all versions of the invention, the active electrode be integrally part of the conductor that extends between the tool terminal and electrode. Likewise, the insulating collar/collars around the base/bases of the active electrode may be formed from a component/components separate from the component that functions as the insulator around the conductor that extends to the active electrode. Similarly, the insulating collar/collars of this invention may have a geometry other than that of a ring. For example, in some versions of the invention, the collar may have a frusto-conical shape.

Likewise, in loop versions of the invention a single conductor may serve as the member that establishes the connection between the ends of the loop electrode and the associated handle terminal.

In some versions of the invention, the bore in the return electrode through which the active electrode extends may not be a bore defined by a completely circumferential internal wall in the return electrode. In these versions of the invention, a section of, if not the whole of, this bore may be a groove or a slot that extends along an outer surface of the return electrode. Here, the conductor leading up to the active electrode is seated in this groove or slot.

In some versions of the invention, control buttons, not illustrated, on the handle 42 allow the practitioner to, with one hand both position the tool and regulate its actuation.

Thus, it is an object of the appended claims to cover all such variations and modifications that come within the true spirit and scope of this invention.

What is claimed is:

1. An electrosurgical tool, said tool including
a handle:
a shaft that extends forward from said handle, said shaft having a distal end;
a first electrode that extends forward from the distal end of said shaft, said first electrode formed from electrically conductive material and having an exposed surface, the surface having at least one section with a substantially linear profile, said first electrode being formed with a bore that opens into the section of said electrode with a substantially linear profile;
a second electrode having a section disposed in the bore of the first electrode and a section that extends out of the bore and forward of the section of said first electrode with a substantially linear profile; and
a sleeve formed from electrically insulating material disposed around at least the section of said second electrode disposed in the bore of said first electrode.

2. The electrosurgical tool of claim 1, wherein:
said first electrode has a front surface that is the distalmost surface of said first electrode, said front surface being formed so as that extending longitudinally along one axis the surface has a substantially linear profile; and
said first electrode is formed so that the bore opens into the front surface so that said second electrode extends forward from the front surface.

3. The electrosurgical tool of claim 1, wherein:
said first electrode has a longitudinal axis that extends between the distal and proximal end of said electrode and a distalmost surface that extends at an angle to the longitudinal axis, the distal most surface having a substantially linear profile; and
said first electrode is formed so that the bore opens into the distalmost surface so that the second electrode extends forward from the first electrode distalmost surface.

4. The electrosurgical tool of claim 1, wherein:
said first electrode has a longitudinal axis that extends between the distal and proximal ends of said electrode and a distalmost surface that extends at an angle to the longitudinal axis, the distalmost surface having a substantially linear profile; and
said first electrode is formed so that the bore opening into the distalmost surface and is centered along the longitudinal axis of said first electrode so that the second electrode extends forward from the first electrode distalmost surface.

5. The electrosurgical tool of claim 1, wherein:
said first electrode has a longitudinal axis that extends between the distal and proximal ends of said electrode and a distalmost surface that extends at an angle to the longitudinal axis, the distalmost surface having a substantially linear profile;
said first electrode is formed so as to have two bore openings that are spaced apart from each other and that open into the distalmost surface of said electrode; and
said second electrode extends out of one of the first electrode bores, forward of said first electrode, bends back to said first electrode and back into a second one of the first electrode bores.

6. The electrosurgical tool of claim 1, wherein:
said first electrode has a longitudinal axis that extends between the distal and proximal ends of said electrode and a distalmost surface that extends at an angle to the longitudinal axis, the distalmost surface being substantially linear, said first electrode being further formed to curve proximally and outwardly away from opposed sides of the distalmost surface; and
said first electrode being further formed so that the bore opens into the distalmost surface so that said second electrode extends forward from the first electrode distalmost surface.

7. The electrosurgical tool of claim 1, further including a non electrically conductive collar disposed around the section of said second electrode that is adjacent said first electrode, said collar extending above the adjacent surface of said first electrode and radially outwardly beyond said second electrode.

8. The electrosurgical tool of claim 1, wherein said sleeve extends forward beyond said first electrode so as to extend over a portion of said second electrode that extends beyond said first electrode.

9. The electrosurgical tool of claim 1, wherein:
said first electrode is formed from material having a relatively high thermal conductivity; and
said second electrode is formed from material having a relatively low thermal conductivity.

10. The electrosurgical tool of claim 1, wherein the section of said first electrode with a substantially linear profile has a length of at least 0.5 mm.

11. The electrosurgical tool of claim 1, wherein the section of said first electrode with a substantially linear profile has a minimum radius of curvature of 3.8 mm.

12. The electrosurgical tool of claim 1, wherein said first electrode is formed is formed so that at least a portion of the bore is located completely within said first electrode.

13. The electrosurgical tool of claim 1, wherein said shaft has a proximal section that is spaced from the distal ends, the proximal section functioning as said handle.

14. An electrosurgical tool, said tool including:
a handle;
a shaft that extends forward from said handle, said shaft having a distal end;
a first electrode that extends forward from the distal end of said shaft, said first electrode formed from electrically conductive material and having an exposed surface, the surface having at least one section with a substantially linear profile, said first electrode being formed with a bore that opens into the exposed surface;
a second electrode having a section disposed in the bore of the first electrode and a section that extends out of the bore and forward of the section of said first electrode with a substantially linear profile;
a sleeve formed from electrically insulating material disposed around at least the section of said second electrode disposed in the bore of said first electrode; and
a collar formed from electrically insulating material that is disposed around a section of said second electrode adjacent said first electrode, said collar extending forward of the exposed surface of said first electrode and beyond an outer perimeter of said second electrode.

15. The electrosurgical tool of claim 14, wherein said sleeve extends forward of the exposed surface of said first electrode so as to function as said collar.

16. The electrosurgical tool of claim 14, wherein:
said first electrode is formed with two bore that have spaced openings to the exposed surface of said first electrode;
said second electrode is shaped to have an extend out of a first one of the first electrode bores, project forward of the exposed surface of said first electrode, back toward the first electrode and into a second one of the first electrode bores;
a said collar is disposed over each section of said second electrode that extends out of one of the first electrode bores.

17. An electrosurgical tool, said tool including:
a handle;
a shaft that extends from said handle, said shaft having a distal end that is spaced from said handle;
a first electrode formed from electrically conductive material that extends forward from said shaft, said first electrode having: an exposed face that, along at least one axis, has a substantially linear profile; and bore that extends at least partially through said electrode, said bore opening into the portion of the exposed face having the substantially linear profile;
a second electrode formed from electrically conductive material, said second bore having a section disposed in the first electrode bore and a section that extends forward from the exposed face of said first electrode;
a sleeve formed from electrically insulating material disposed around at least the section of said second electrode disposed in the bore of said first electrode; and
a collar formed from electrically insulating material that is disposed around a section of said second electrode adjacent said first electrode, said collar extending forward of the exposed surface of said first electrode and beyond an outer perimeter of said second electrode.

18. The electrosurgical tool of claim 17, wherein:
said first electrode is formed with two bore that have spaced openings to the exposed surface of said first electrode;
said second electrode is shaped to have an extend out of a first one of the first electrode bores, project forward of the exposed surface of said first electrode, back toward the first electrode and into a second one of the first electrode bores;
a said collar is disposed over each section of said second electrode that extends out of one of the first electrode bores.

19. The electrosurgical tool of claim 17, wherein said first electrode has a distalmost section, the section being the section of said first electrode with a substantially linear profile.

20. The electrosurgical tool of claim 17, wherein:
said first electrode has a distalmost section, the section being the section of said first electrode with a substantially linear profile; and
said first electrode is further formed so that the exposed face curves proximally and outwardly away from the distalmost section on opposed sides of the distalmost section.
